(19) United States
(12) Patent Application Publication
(10) Pub. No.: US 2007/0073310 A1
(43) Pub. Date: Mar. 29, 2007

(54) Method for joining medical devices

(75) Inventors: Dharmendra Pal, Wilmington, MA (US); Fred T. Parker, Unionville, IN (US); Jeffry S. Melsheimer, Springville, IN (US)

Correspondence Address: BRINKS HOFER GILSON & LIONE/INDY/COOK, ONE INDIANA SQUARE, SUITE 1600, INDIANAPOLIS, IN, 46204-2033, US

(73) Assignee: Cook Incorporated, Bloomington, IN (US)

(21) Appl. No.: 11/524,863

(22) Filed: Sep. 21, 2006

Related U.S. Application Data
(60) Provisional application No. 60/721,696, filed on Sep. 29, 2005.

(51) Int. Cl. A61F 11/00

(52) U.S. Cl. 606/108; 264/248; 264/230

(57) ABSTRACT

A method for joining at least first and second medical devices, and an apparatus comprising joined medical devices. A first tubular structure has a plurality of layers, wherein one of the layers has a portion adapted for joinder to a second tubular structure. The second tubular structure has a portion adapted for joinder to the first tubular structure. The first and second tubular structures are positioned such that the respective adapted portions are aligned for joinder, and the tubular structures are heated such that at least one of the adapted portions melts and flows into engagement with the other adapted portion to form a bond therebetween. The first tubular structure may comprise an inner PTFE layer and an outer layer of a polymer such as nylon or PEBA, and the second tubular structure may comprise a high strength polymer, such as PEEK.

### RELATED APPLICATIONS

This application claims priority to U.S. Provisional Patent Application No. 60/721,696, entitled “SYSTEM AND METHOD FOR JOINDER OF MEDICAL DEVICES,” filed Sep. 29, 2005, which is incorporated herein by reference in its entirety.

### BACKGROUND

1. Technical Field

The present invention relates to a method for joining medical devices, and to an apparatus comprising joined medical devices. More particularly, the invention relates to a method for joining a hollow tubular structure to a multi-layer medical device, such as an introducer sheath, and to an apparatus formed by the method.

2. Background Information

Medical introducer devices, such as sheaths, catheters and similar structures (collectively referred to as “introducers”), are in widespread use in modern medical practice as conduits to provide percutaneous access to the vascular system. Frequently, introducers are utilized to introduce a liquid medicament to a site well within the vascular system of a patient. Introducers are also commonly used to introduce an implantable medical device, such as a stent, to a suitable deployment site within the vascular system of a patient. On other occasions, introducers are used to withdraw a body fluid or a solid, such as an embolus or a thrombus, from a target site within the vasculature.

Typically, an introducer is a multi-layer structure comprising an inner liner formed of a lubricious polymeric material, such as PTFE, and an outer layer formed of a polymeric material, such as nylon or a polyether block amide. A reinforcing structure, such as a helical coil, is typically provided between the inner and outer layers. The outer jacket is joined to the inner liner by melting the outer jacket such that it adheres to a roughened outer surface of the PTFE liner through turns of the coil. Although introducers may be constructed to have virtually any size necessary to accomplish a desired operation, introducers typically have a diameter between about 5 and 12 French, and a length between about 40 and 90 cm. One example of such an introducer sheath is provided in U.S. Pat. No. 5,380,304, incorporated by reference herein.

Other known introducers may be formed as multi-layer structures utilizing different compositions than those described above. In addition, some known introducers are structured such that the properties of the introducer vary along the axial length of the structure. One example of an introducer of this type has a higher (i.e. harder) durometer at one axial end of the introducer, such as the proximal end, and a lower (i.e., softer) durometer at the other end of the introducer, such as the distal end. Introducers may be provided with additional features, such as a radiopaque marker and/or a hard distal tip, to enhance desired properties of the introducer for a particular use.

On some occasions, it may be desired to modify portions of an introducer such that the introducer can be used to perform functions that may not otherwise be possible, or that may be possible only in limited circumstances. One example involves the modification of one or both of the axial ends of the introducer. This may be beneficial, for example, when it is desired to extend the length of the introducer beyond the length of the particular introducer at hand. Such an expanded length introducer may be desired when, for example, the physician desires an introducer having a greater length that the introducers available to her. Additionally, such an extended length may be beneficial when an extended portion need not include all of the properties of the original introducer, or alternatively, when an extended portion is desired that has properties not provided by the original introducer. Non-limiting examples of such properties that may be of interest include a difference in the durometer of the extended portion when compared to the original introducer, or a difference in the tensile strength when compared to that of the original introducer. In addition, it may be desired to provide an extended segment having features not present on the original introducer. In this instance, a segment can be joined to an axial end of the introducer to provide such features. Examples of such features may include the addition of a segment having a radiopaque marking band, or a segment having a different diameter than the remainder of the introducer.

### SUMMARY

The present invention describes a method for joining tubular structures, and a medical introducer apparatus formed by joining two or more tubular structures.

In one form thereof, the invention comprises a method for joining first and second tubular medical structures. The first tubular structure has a plurality of layers, wherein one of the layers has a portion adapted for joinder to a second tubular structure. The second tubular structure has a portion adapted for joinder to the first tubular structure. The first and second tubular structures are positioned such that the respective adapted portions are aligned for joinder, and the tubular structures are heated such that at least one of the adapted portions melts and flows into engagement with the other adapted portion to form a bond therebetween. In one embodiment, the first tubular structure may comprise an inner PTFE layer and an outer layer of a polymer such as nylon or PEBA, and the second tubular structure may comprise a high strength polymer, such as PEEK.

In another form thereof, the invention comprises a medical introducer apparatus comprising a first tubular structure and a second tubular structure. The first tubular structure comprises a plurality of layers, and has a first joinder element at an axial end thereof. The second tubular structure also has a joinder element at an axial end thereof The joinder element of the first tubular structure is melt bonded with the joinder element of the second tubular structure. In an alternative embodiment, the first tubular structure comprises a joinder element at each end thereof, and the apparatus includes respective second and third tubular structures at opposite ends thereof. The fist tubular structure may comprise an inner PTFE layer and an outer layer of a polymer such as nylon or PEBA. At least one of the second and third tubular structures may comprise a high strength polymer, such as PEEK.

### BRIEF DESCRIPTION OF THE DRAWINGS

FIG. 1 depicts a prior art flexible, kink-resistant introducer sheath shown in combination with a dilator and a connector valve;

FIG. 2 is a partially sectioned view of the prior art introducer sheath of FIG. 1;

FIG. 3 illustrates a sectional view of an apparatus formed by the joinder of an introducer and a tubular structure, according to an embodiment of the present invention;

FIG. 4 illustrates a sectional view of an introducer used to form the apparatus of FIG. 3, prior to bonding with the tubular structure;

FIG. 5 illustrates a tubular structure that is joined to the introducer to form the apparatus of FIG. 3;

FIG. 6 illustrates a sectional view of an alternate embodiment of an apparatus formed by the joinder of a tubular structure to each axial end of an introducer;

FIG. 7 illustrates a tubular structure having a flared axial end for joinder to an introducer; and

FIG. 8 illustrates a tubular structure having a slitted axial end for joinder to an introducer.

### DETAILED DESCRIPTION OF THE DRAWINGS AND THE PRESENTLY PREFERRED EMBODIMENTS

For the purposes of promoting an understanding of the principles of the invention, reference will now be made to the embodiments illustrated in the drawings, and specific language will be used to describe the same. It should nevertheless be understood that no limitation of the scope of the invention is thereby intended, such alterations and further modifications in the illustrated device, and such further applications of the principles of the invention as illustrated therein being contemplated as would normally occur to one skilled in the art to which the invention relates.

In the following discussion, the terms “proximal” and “distal” will be used to describe the opposing axial ends of the introducer apparatus, as well as the axial ends of various component features. The term “proximal” is used in its conventional sense to refer to the end of the apparatus, or component thereof, that is closest to the operator during use of the apparatus. The term “distal” is used in its conventional sense to refer to the end of the apparatus, or component thereof, that is initially inserted into the patient, or that is closest to the patient.

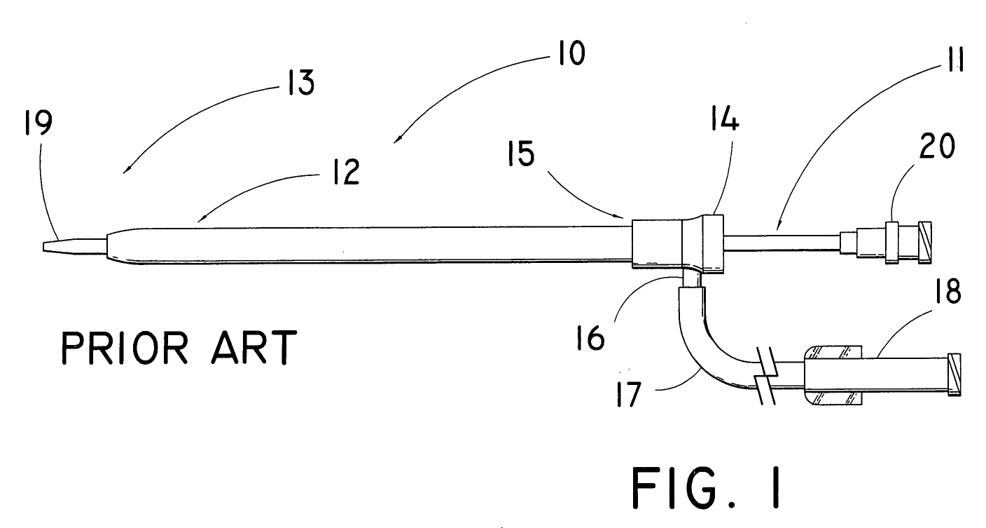

FIG. 1 depicts an illustrative flexible, kink-resistant, introducer sheath 10 of the type described and shown in the incorporated-by-reference U.S. Pat. No. 5,380,304. The prior art introducer shown in FIG. 1 represents one example of an introducer that may be advantageously joined to another structure according to the system and method of the present invention.

Prior art introducer sheath 10 includes an outer tube 12, which is provided with a tapered distal end 13, and a flared proximal end 15. The introducer 10 is shown in combination with an optional tapered dilator 11 and an optional connector valve 14. Dilator 11 extends longitudinally through the inner passageway of the sheath. Dilator 11 may include a tapered distal end 19 for accessing and dilating a vascular access site over a wire guide in well-known fashion. A conventional male Luer lock connector hub 20 may be attached at the proximal end of the dilator for connection to a syringe or other medical apparatus. Connector valve 14 may be attached about the flared proximal end of the sheath, and may include a conventional silicone valve disk (not shown) for preventing the backflow of fluids therethrough. Connector valve 14 may also include a side arm 16, having a conventional tube 17 and male Luer lock connector 18 engaged thereto for introducing and/or aspirating materials, such as fluids and solids, through the introducer.

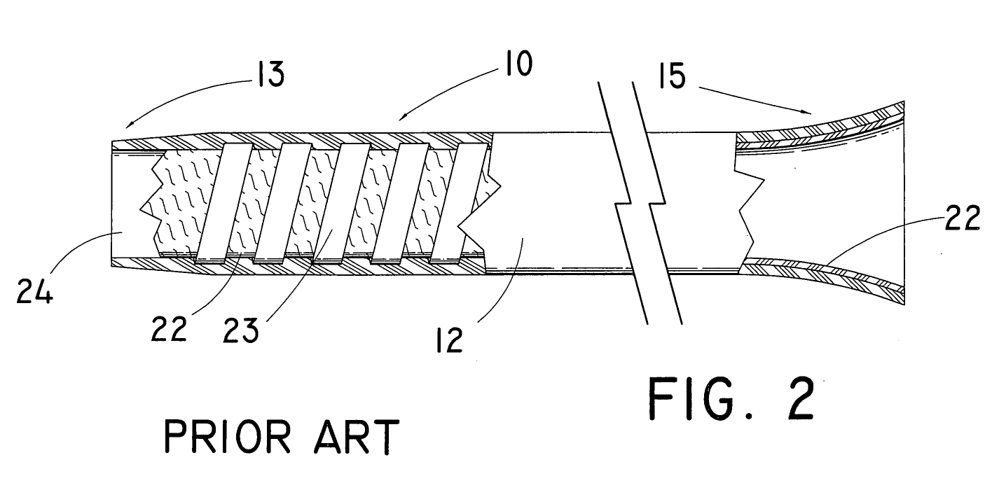

FIG. 2 depicts a partially sectioned view of prior art introducer sheath 10. The optional dilator and connector valve have been omitted from this figure. Introducer 10 comprises an inner tube 22, and a coil 23 that is wound or otherwise fitted around inner tube 22. Outer tube 12 is mechanically connected to a roughened outer surface of inner tube 22 through the spacings of the coil. Conventional inner tube 22 is typically formed of a lubricious material, such as PTFE. The outer surface of the inner tube is generally chemically etched in well-known manner to provide a roughened surface. Roughening this outer surface enhances the engagement between the inner surface of outer tube 12 and the outer surface of inner tube 22.

Inner tube 22 will generally have a uniform inner diameter that extends the entire length of the passageway 24 that extends through the inner tube. In this manner, the largest possible diameter catheter, stent, dilator or other interventional device can be passed through the sheath. When dealing with intravascular devices it is generally desirable to utilize a device having the largest possible inner diameter, and the smallest possible outer diameter, that is sufficient to achieve the intended purpose. Thus, the thickness of the PTFE tube is limited to the greatest extent possible, while maintaining the necessary structural integrity to prevent the turns of coil 23 from protruding into inner tube passageway 24, and to otherwise maintain the necessary strength to accomplish the intended purpose.

Coil 23 is typically compression fitted or wound over inner tube 22. Coil 23 is typically as a stainless steel flat wire coil, but may also be formed of other compositions, such as a super-elastic alloy such as nitinol. In addition, coils having cross-sectional dimensions other than flat wire, such as round wire, can also be used. In the prior art configuration shown in FIG. 2, the flat wire coil includes substantially uniform spacings of equal width between the turns of the coil, and the coil turns are spaced at a constant pitch. In most cases, smaller diameter sheaths are provided with coil turns closer together, while larger diameter sheaths are provided with coil turns spaced apart a greater distance. Increasing the space between the coil turns generally increases the flexibility of a sheath, while decreasing the space between coil turns decreases the flexibility of the sheath.

The coil is typically spaced from the distal and proximal ends of the inner tube. This spacing permits tapering and flaring of the respective distal and proximal ends of the sheath if desired. In a conventional configuration in which a valve is attached at the proximal end of the sheath and a tapered tip formed at the distal end, the coil may be terminated between about 0.5 and 5.0 inches (1.27 and 12.7 cm), more preferably about 1.2 inches (3.1 cm), from the proximal end of the sheath, and between about 0.1 and 2.0 inches (0.25 and 5.1 cm), more preferably about 0.8 inch (2 cm), from the distal end.

Outer tube 12 can be formed of any well-known polymer commonly used for such purpose. Typically, outer tube 12 comprises a heat formable polyamide material, such as nylon, or a polyether block amide (PEBA). The material of outer tube 12 will generally be sufficiently flexible so that the introducer can navigate the tortuous pathways encountered in the vascular system. If desired, different axial portions of the outer tube are provided with different durometers so that the flexibility of the introducer can vary in a desired fashion along its length.

During assembly of prior art sheath 10, inner liner 22 is typically fitted over a suitably-sized mandrel, and coil 23 is compression fitted or wound around inner liner 22. Outer tube 12 is disposed or positioned over the coil and the inner liner, and the entire assembly is enveloped in a conventional heat shrink tube. The entire assembly is baked in an oven, whereupon the outer tube begins to melt. A melted portion of the outer tube flows between the coil turns in well known fashion, and is mechanically connected to the roughened outer surface of the inner liner. The heat-formable outer tube is essentially self-leveling after heating, which provides a uniform outer diameter to the outer tube. Once the outer tube has shrunk onto the roughened surface of the inner liner and cooled, the heat shrink tube is split and cut off, and the mandrel is removed. The process for preparation of a conventional introducer sheath described above is well-known, and is further described, for example, in U.S. Pat. No. 5,380,304.

Various other materials may be utilized for the inner, outer, and heat shrink tubes, which materials are well known in the art. Additionally, various other reinforcements, such as a braided reinforcement, may be utilized, which reinforcements are also well known in the art.

One facet of the present invention involves joining a hollow tubular member to an axial end of an introducer that may be of the type described hereinabove. Although the description herein generally refers to introducers, those skilled in the art will appreciate that the teachings of the invention can be extended to the joinder of other tubular medical devices, which joinder is considered within the scope of the invention.

There are many reasons why joining an additional tube to an existing introducer may be beneficial. For example, it may be desired to expand the length of the introducer. This is not only useful when it is desired to lengthen the introducer, but may also be particularly useful when the joined portion need not include all of the properties of the original introducer, or alternatively, when the joined portion includes properties not present in the original introducer. For example, a joined portion need not always include a reinforcing layer. In such instances, a simple tubular structure may be joined to the original introducer that does not include the reinforcement. Although such joined structures will not normally exhibit the kink resistance or torque properties that may be provided by an introducer having a coil or a braided reinforcement, in many instances such properties are not necessary for a joined segment, and their omission does not cause undue inconvenience. Alternatively, a joined portion can provide additional properties that are not provided by the original introducer. Examples of such additional properties include a higher or lower durometer, torque, tensile strength, etc., that are not exhibited by the original introducer length. Those skilled in the art may readily recognize other circumstances in which it may be advantageous to provide an introducer with an axial end portion having a different length and/or different properties when compared to the main body portion of the introducer.

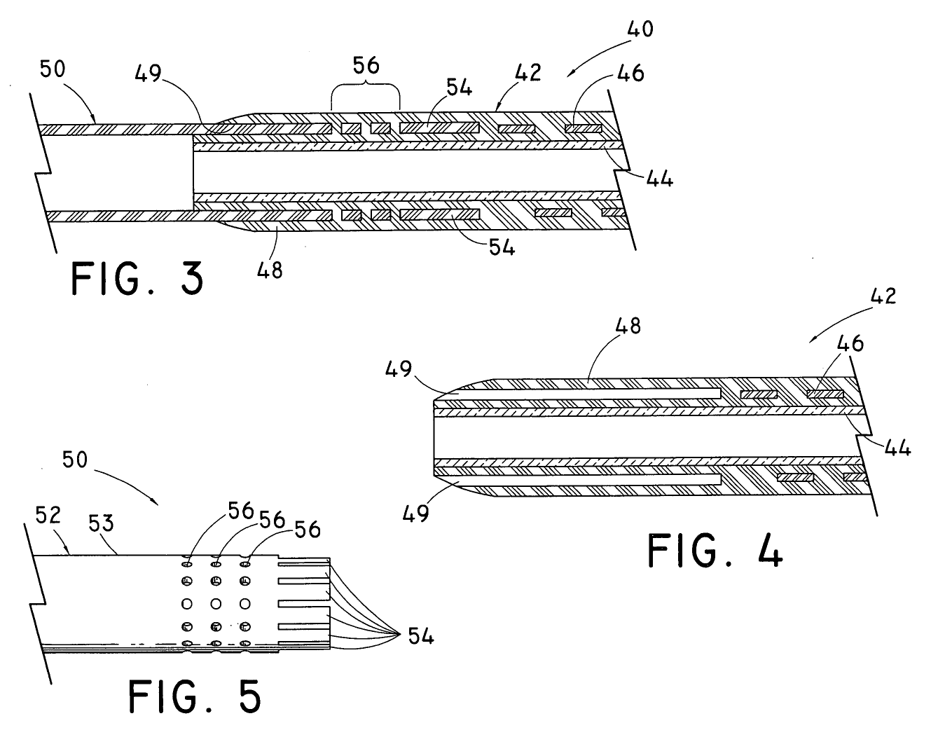

One example of a joined apparatus 40 according to the present invention is illustrated in FIG. 3. In this embodiment, joined apparatus 40 is formed by joining an introducer 42 to a tubular structure 50. One example of an introducer 42 prior to joinder is shown in FIG. 4, and an example of a tubular structure 50 is shown in FIG. 5.

In the embodiment shown in FIGS. 3 and 4, introducer 42 may comprise a conventional introducer such as the introducer of the '304 patent described above. Introducer 42 comprises an inner liner 44, a coil reinforcement 46, and an outer jacket 48, each of which may be formed utilizing conventional compositions and according to conventional techniques. As one non-limiting example, inner liner 44 may comprise PTFE, the coil 46 may comprise stainless steel, and the outer layer 48 may comprise nylon or PEBA. The outer layer attaches to the outer surface of the inner PTFE liner through the coil turns. In this embodiment, a channel 49 is cut or otherwise formed at an axial end of introducer 42. Channel 49 will ordinarily extend between an axial termination point of the coil 46 and the axial end of outer jacket 48. Preferably, channel 49 extends axially inwardly into the introducer from the axial end a distance of about 0.5 to about 2 cm, although longer, or shorter, channel lengths may be provided if desired. Preferably, channel 49 is cut into the end of outer layer 48 at substantially the same radial distance from the interior of introducer 42 as coil 46.

One non-limiting example of a tubular structure 50 of a type suitable for joining with introducer 42 is shown in FIG. 5. In this example, tubular structure 50 comprises a generally tubular body 52 having an axial end 53. A plurality of fingers 54 extend in the axial direction from axial end 53, and are arranged circumferentially around axial body end 53. A plurality of apertures 56 are provided along a portion of the circumference of axial end 53, and extend through a side wall of axial end 53 into the interior of the tubular body.

Tubular structure 50 can be formed of virtually any medical grade composition that is capable of joining with the introducer. Preferably, tubular structure 50 comprises a high strength, high melting polymer. One preferred example is polyether ether ketone (PEEK). PEEK is generally capable of flexing as required, and is hard enough to withstand stresses encountered as it is passed through tortuous turns in the vasculature. PEEK also generally will have a higher strength than the introducer body 42, and thus imparts a greater strength to the axial end upon joinder with the introducer. Furthermore, PEEK exhibits favorable kink resistance and pushability. However, those skilled in the art will appreciate that other well-known tubular compositions, such as stainless steel, nitinol, brass, copper, and various polymers having desirable properties may also be used to form tubular structure 50. Preferably, such compositions will have appropriate sterilizability for use in medical applications, and will exhibit appropriate incompressibility as desired for a particular use.

As shown in FIG. 3, tubular structure 50 is joined to introducer 42 by inserting axial end 53 of tubular body 52 within channel 49 of the introducer. The apparatus is exposed to a suitable heat source, such that outer layer 48 melts and flows between fingers 54 and through apertures 56 of tubular structure 50. The melted material that seeps between fingers 54 and through apertures 56 securely bonds tubular structure 50 to introducer 42, thereby forming the joined apparatus 40.

The outer jacket 48 of introducer 42 typically has a lower melting point than the melting point of the inner PTFE liner, and of the tubular structure 50. As a result, outer jacket 48 melts and flows as described, whereas inner liner 44 and tubular structure 50 maintain their structural integrity upon heating. The heating may take place in a heat shrink tube of the type that is commonly used in the art for bonding an outer jacket of a sheath to an inner liner. Those skilled in the art can readily select appropriate heat sources and conditions for melting of an introducer to bond to another tubular structure for use in accordance with the teachings of the present invention.

Although preferred embodiments of the inventive apparatus and method for preparing the apparatus have been described, other apparatuses and methods may be utilized within the scope of the invention. The method described above teaches the formation of apparatus 40 by joinder of an existing introducer 42 to a tubular structure 50. However, apparatus 40 need not necessarily be formed by joining tubular structure 50 to an existing introducer 42. Rather, in another embodiment, tubular structure 50 may be joined to introducer 42 at the time of formation of introducer 42. In this case, tubular structure 50 may be bonded to introducer 42 during the same heat shrink operation utilized to bond inner liner 44 and outer jacket 48.

In this alternative embodiment, inner liner 44 is fitted over a mandrel, and coil 46 is compression fitted or wound around the inner liner as before. Outer layer 48 is disposed or otherwise positioned over the coil and the inner liner. Axial end 53 of tubular body 52 is inserted into channel 49, and the entire assembly is placed in a conventional heat shrink tube. The envelope is placed in an oven which has been heated to a temperature sufficient for melting outer layer 48. As the outer layer melts, the material flows between the coil turns to bond the outer layer to the roughened outer layer of the PTFE inner liner in well-known fashion. At the same time, the melted material flows between fingers 54, and through apertures 56, of tubular body 52, thereby bonding tubular structure 50 to the introducer 42. The entire assembly is removed from the mandrel and allowed to cool. The heat shrink tube is then cut away, leaving the joined apparatus 40.

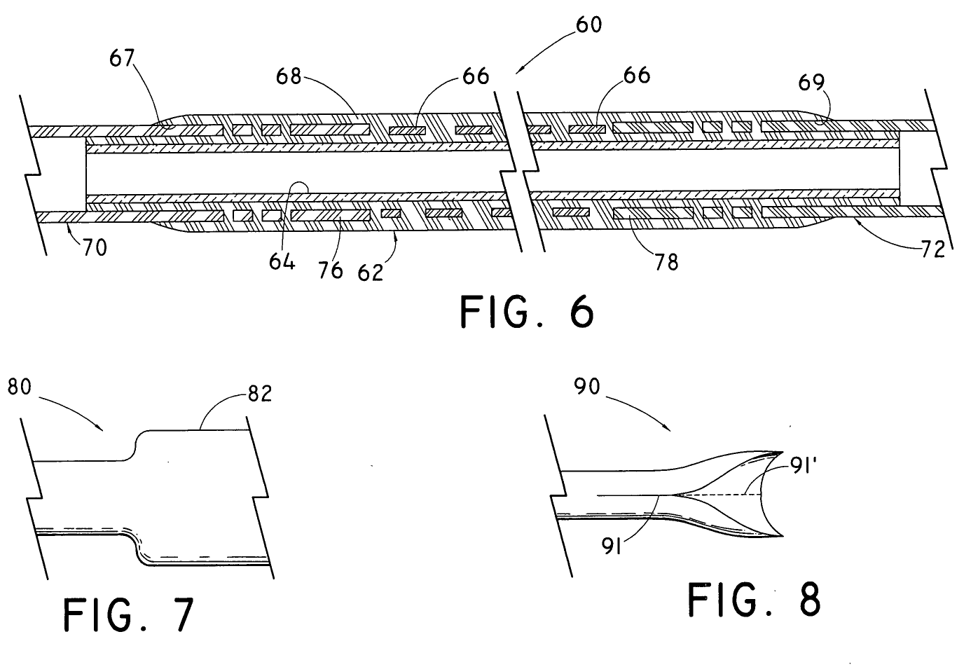

Although the examples provided above describe the joinder of a tubular structure to a single axial end of the introducer, FIG. 6 illustrates an alternative apparatus 60 wherein a separate structure is joined to each axial end of introducer 62. Introducer 62 includes inner liner 64, coil 66, and outer jacket 68, as before. In this embodiment, introducer 62 includes channels 67, 69 at each axial end. Tubular structures 70, 72 having the same, or different, compositions may be joined to respective axial ends of the introducer. Tubular structures 70, 72 may include fingers 76, 78, and apertures as before, for use in joining the structures 70, 72 to respective axial ends of introducer 62. In this embodiment, apparatus 60 can be provided with, for example, a high durometer structure joined at one axial end and a lower durometer structure joined at the other axial end. Similarly, other structures can be joined to distinguish the properties at one axial end of the resulting apparatus, from the properties of the opposite axial end.

The examples provided above describe a system of joinder wherein the introducer includes a slot or channel of a suitable size and shape for receiving an axial end of the tubular structure to be joined. Although this arrangement provides a very suitable means of joining tubular structures, alternative arrangements are also within the scope of the invention. For example, although the examples describe the use of both fingers and apertures on the tubular structures, it is not necessary that both fingers and apertures be provided on each such structure, and in many instances, a single mechanism for joining the structures will be sufficient. Similarly, although the examples describe the use of a plurality of fingers and/or apertures on each of the tubular structures, it is not always necessary to provide a plurality of such features. In many instances a single finger, aperture or other structure having a shape and composition to provide sufficient joinder with the introducer to accomplish the desired purpose may be utilized. Those skilled in the art will appreciate that various other shapes and configurations may be substituted for those described, each of which is considered within the scope of the invention.

The examples provided above also describe a system of joinder wherein the introducer includes the slot or channel, and the structure to be received within the slot or channel is provided on the tube. However, this arrangement could be reversed if desired. In other words, the tubular structure to be joined to the introducer could be provided with the slot or channel, and the axial end(s) of the introducer could be provided with the specific features utilized for joinder, such as the fingers or apertures.

The tubular structure to be joined need not have the same inner diameter as the introducer. In some instances, however, it may be desirable to modify the tubular structure and/or the introducer such that they have virtually the same inner diameter. Those skilled in the art will appreciate that there are numerous ways in which one or the other of these elements may be modified to alter its inner diameter such that it matches the inner diameter of the element to be joined. One way of doing this is to create a flared or swaged end of one of the elements. FIG. 7 illustrates an example of a tubular structure 80 having flared end 82. Flaring or swaging of the end of a tubular structure is a well-known concept, and standard tools are readily available for such purposes. Apertures and/or fingers can be formed in flared end 82 to enhance joinder to the introducer.

FIG. 8 illustrates another example wherein the inner diameter of a tubular structure 90 may be modified for joinder to an introducer. In this example, an axial end of structure 90 is split on along line 91, 91′. Split line 91 is illustrated in the figure and line 91′ is on the opposite side of line 91 (shown in broken lines).

It is therefore intended that the foregoing detailed description be regarded as illustrative rather than limiting, and that it be understood that it is the following claims, including all equivalents, that are intended to define the spirit and scope of this invention.

1. A method for joining tubular medical structures, comprising:
providing a first tubular structure, said first tubular structure comprising a plurality of layers, a first one of said layers having a portion adapted for joinder to a second tubular structure;
providing a second tubular structure, said second tubular structure having a portion adapted for joinder to said adapted portion of said first tubular structure;
positioning said first and second tubular structures such that said respective adapted portions are aligned for joinder; and
heating said tubular structures such that at least one of said adapted portions melts and flows into engagement with the other adapted portion to form a bond therebetween.

2. The method of claim 1, wherein the adapted portion of said first tubular structure has a melting point lower than the melting point of the adapted portion of said second tubular structure, such that upon heating of said tubular structures, said adapted portion of said first tubular structure melts and flows into the adapted portion of the second tubular structure to form said bond.

3. The method of claim 2, wherein said plurality of layers of said first tubular structure includes said first layer and a second layer, said second layer comprising an inner liner, and said first layer circumferentially surrounding said inner liner.

4. The method of claim 3, wherein said first tubular structure further comprises a reinforcing layer.

5. The method of claim 4, wherein said inner liner of said first tubular structure comprises PTFE, said reinforcing layer comprises a coil, and said layer surrounding said inner liner comprises nylon.

6. The method of claim 5, wherein said second tubular structure comprises a high strength polymer.

7. The method of claim 6, wherein said polymer comprises polyether ether ketone.

8. The method of claim 2, wherein said adapted portion of said first tubular structure comprises a channel, and said adapted portion of said second tubular structure comprises at least one member sized and shaped to be received in said channel.

9. The method of claim 8, wherein said channel has a length of between about 0.5 and 2 cm.

10. The method of claim 8, wherein said member comprises at least one of a plurality of axially-extending fingers and a plurality of apertures.

11. The method of claim 8, wherein said member comprises at least one of an axially-extending finger and an aperture.

12. The method of claim 2, further comprising placing said first and second tubular structures having said portions aligned for joinder in a heat shrink receptacle, and heating said tubular structures in said heat shrink receptacle.

13. The method of claim 2, wherein said first layer has first and second axial ends, said first layer having said portion adapted for joinder to said second tubular structure at one of said axial ends, and having a portion adapted for joinder to a third tubular structure at the other axial end, said portion adapted for joinder to said third tubular structure having a melting point lower than a melting point of a portion of said third tubular structure adapted for joinder to said portion of said first tubular structure, said method further comprising:
positioning said first and third tubular structures such that said other axial end of said first layer is aligned for joinder with said adapted portion of said third tubular structure; and
heating said first, second and third tubular structures such that said adapted portions of said respective axial ends of said first layer of said first tubular structure melt and flow into the respective adapted portions of the second and third tubular structures.

14. The method of claim 13, further comprising placing said first, second and third tubular structures having said portions aligned for joinder in a heat shrink receptacle, and heating said tubular structures in said heat shrink receptacle.

15. A medical introducer apparatus, comprising:
a first tubular structure and a second tubular structure, said first tubular structure comprising a plurality of layers and having a first joinder element at an end thereof, said second tubular structure comprising a joinder element at an end thereof, wherein said joinder element of said first tubular structure is melt bonded with said joinder element of said second tubular structure.

16. The medical introducer apparatus of claim 15, wherein said joinder element of said first tubular structure comprises a channel formed in one of said layers, and said joinder element of said second tubular structure comprises a member received in said channel.

17. The medical introducer apparatus of claim 15, wherein said first tubular structure comprises a joinder element at each end thereof, said apparatus further comprising a third tubular structure having a joinder element at an end thereof, wherein the joinder element at the other end of said first tubular structure is fixedly engaged with the joinder element of said third tubular structure.

18. The medical introducer apparatus of claim 17, wherein said joinder elements of said first tubular structure comprise respective channels formed at opposite ends of one of said layers, and said joinder elements of said second and third tubular structures comprise members fixedly received in said respective channels.

19. The medical introducer apparatus of claim 15, wherein said second tubular structure comprises a high strength polymer.

20. The medical introducer apparatus of claim 18, wherein at least one of said second and third tubular structures comprises a high strength polymer.
